(19) United States
(12) Patent Application Publication
(10) Pub. No.: US 2025/0179415 A1
(43) Pub. Date: Jun. 5, 2025

(54) AN APPARATUS FOR SEPARATING MICRO-SWIMMERS

(71) Applicant: Monash University, Clayton (AU)

(72) Inventors: Farin Yazdan Parast, Clayton (AU); Moira K. O'Bryan, Melbourne (AU); Reza Nosrati, Clayton (AU)

(21) Appl. No.: 18/719,733

(22) PCT Filed: Dec. 14, 2022
(86) PCT No.: PCT/AU22/51502

§ 371 Date: Jun. 13, 2024

(30) Foreign Application Priority Data
Dec. 14, 2021 (AU) 2021904063

(51) Int. Cl. C12M 1/00; C12M 3/00; C12M 3/06

(52) U.S. Cl. CPC C12M 47/04; C12M 21/06; C12M 23/16

(57) ABSTRACT

The present invention is directed to an apparatus for separating micro-swimmers. The apparatus comprises a chamber having an inlet sub-chamber and an outlet sub-chamber, a plurality of microchannels disposed between the inlet sub-chamber and the outlet sub-chamber, a passage control mechanism for controlling the passage of micro-swimmers from the inlet sub-chamber to the outlet sub-chamber via the microchannels. The passage control mechanism is configurable to operate in a first condition in which passage of micro-swimmers from the inlet sub-chamber to the outlet sub-chamber are prevented, and a second condition in which passage of micro-swimmers from the inlet sub-chamber to the outlet sub-chamber via the microchannels are permitted.

### TECHNICAL FIELD

The present invention relates to an apparatus for separating micro-swimmers, a method of using the apparatus and a method of separating micro-swimmers. In particular, the micro-swimmers include sperm, although the scope of the invention is not necessarily limited thereto.

### BACKGROUND OF INVENTION

Infertility is a global health concern, affecting one in six couples in Australia and as high as 70 million couples worldwide. Male infertility issues, including low sperm count, lack of viability, poor motility, abnormal morphology, and/or low DNA integrity contribute to a total of 45% of infertility cases. Assisted reproductive technologies (ART) have been developed over the past 40 years to treat infertility. ART includes intracytoplasmic sperm injection (ICSI), in vitro fertilization (IVF) and intrauterine insemination (IUI). In ICSI, as the most invasive treatment method, an individual sperm is selected and injected directly into an egg. In IVF or IUI, a subpopulation of pre-selected sperm is introduced close to the egg to achieve fertilization.

The quality of selected sperm is crucial to ART, contributing significantly to the treatment success rate, live-birth rate and offspring health. However, the current clinical sperm selection practices are highly manual, subjective, and prone to operator errors, resulting in suboptimal ART success rate, stagnated at ˜33% per cycle over the past 30 years.

Density gradient centrifugation and swim-up are some of the currently known methods for sperm selection in clinics. In swim-up assay (SU), highly motile sperm swim upward from the raw semen sediment into a fresh sperm media, where they are collected after ˜60 minutes. However, this method is not applicable in the case of asthenozoospermia. In density gradient centrifugation (DGC), sperm are separated based on their density by being forced to cross a viscosity gradient via centrifugation force that could cause sperm DNA damage. Both SU and DGC are time-consuming (taking roughly 30 to 60 minutes in processing time), require multiple preparation steps, depend on the clinician expertise, and differ significantly from the natural selection process in vivo.

Moreover, the relatively long processing time and considerably low retrieval efficiency (i.e. the ratio of separated sperm count to the total number of processed sperm) of these conventional methods have considerably restricted the clinical workflow, especially due to the limited sperm lifetime in vitro. Despite these shortcomings, the clinical sperm selection methods remain almost unchanged over the past 40 years, contributing to the relatively low success rate of treatment cycles.

Some alternative sperm preparation methods have been developed to select sperm based on their morphological characteristics under high magnification, maturity via HA-bounding, and membrane charge using electrophoresis, or by removing apoptotic sperm using magnetic cell separation. However, clinical application of these methods is still limited, mainly due to their highly manual procedures, harmful effect on sperm motility, dependence on clinician expertise and lack of validation.

Microfluidics and lab-on-a-chip methods have provided opportunities for automation and standardization of processes at the single-cell level. Microfluidic platforms have been developed to assist infertility diagnosis and treatment including devices for home-based semen analysis, understanding sperm swimming behaviour, and selecting high-quality sperm. With respect to sperm selection, these platforms use passive strategies based on sperm motility or active strategies using flow, chemical gradient, or acoustic forces to select high-quality sperm. Despite significant improvement in microfluidic sperm selection devices over the past two decades, implementation and translation of these technologies into clinical settings has been considerably slow, mainly due to the lack of scalable microfabrication opportunities and complexity in operation.

Embodiments of the invention may provide an apparatus for separating micro-swimmers such as sperm, a method of using the apparatus and a method of separating micro-swimmers such as sperm which overcomes or ameliorates one or more of the disadvantages or problems described above, or which at least provides the clinician with a useful choice.

A reference herein to a patent document or any other matter identified as prior art, is not to be taken as an admission that the document or other matter was known or that the information it contains was part of the common general knowledge as at the priority date of any of the claims.

### SUMMARY OF INVENTION

According to one aspect of the invention, there is provided an apparatus for separating micro-swimmers, the apparatus comprising
a chamber having an inlet sub-chamber and an outlet sub-chamber,
a plurality of microchannels disposed between the inlet sub-chamber and the outlet sub-chamber,
a passage control mechanism for controlling the passage of micro-swimmers from the inlet sub-chamber to the outlet sub-chamber via the microchannels, the passage control mechanism being configurable to operate in
a first condition in which passage of micro-swimmers from the inlet sub-chamber to the outlet sub-chamber are prevented, and
a second condition in which passage of micro-swimmers from the inlet sub-chamber to the outlet sub-chamber via the microchannels are permitted.

In some applications, micro-swimmers may include sperm. However, micro-swimmers may include any microscopic object with the ability to move in a fluid environment. For example, in some applications, micro-swimmers may include other biological microorganisms, such as bacteria, archaea, protists and micro-animals.

Moreover, the apparatus and methods described herein may be used for variety of purposes, such as (but not limited to) combinations of the following applications: sperm selection, semen purification, high viability selection, high concentration selection, normal morphology selection, high DNA integrity selection, high maturity selection, or improvement in selected population of motile cells in terms of: viability, concentration, DNA integrity, morphology and/or maturity. The apparatus and methods may be used for clinical or home-based semen purification for application in in vitro fertilization (IVF) and intrauterine insemination (IUD).

In some embodiments, the apparatus and methods described herein may be also used for selection of motile microorganisms such as motility mutants Caenorhabditis elegans from non-mutant, Caenorhabditis elegans selection for genetic analysis, selection of motile bacteria for cancer treatment, selection of highly motile Salmonella for tumour dispersion, selection of bacteria for food, water quality management, bioengineering and pharmaceutical industries. Accordingly, it should be noted that the terms such as ‘micro-swimmer separation’ and ‘sperm separation’ as used herein may include any one or more of: selection, purification, rapid selection, and high throughput selection of motile cells or organisms.

The microchannels can take any suitable shape or form. The apparatus may provide a two-dimensional network of microchannels. For example, the microchannels may be distributed across a single plane. In other embodiments, the microchannels may be distributed across multiple planes, or distributed across a three-dimensional volume. For example, the plurality of microchannels may include a three-dimensional network of microchannels.

The microchannels may for regular patterns or arrays. Alternatively, the microchannels may be randomly distributed.

The microchannels can have any regular or irregular cross-sectional shape. For example, the microchannels can have a generally circular cross-section, rectangular cross-section, triangular cross-section, any irregular shaped cross-section, or a combination thereof.

The apparatus may include a channel member defining the plurality of microchannels therein. The channel member may have any suitable regular or irregular shape. For example, the channel member may be generally cylindrical or cuboidal. More specifically, the channel member may have a hollow cylindrical body.

In some embodiments, the channel member may be an assembly (also referred to herein interchangeably as a channel assembly). The channel assembly may comprise a plurality of channel inserts, and a frame defining a plurality of windows for receiving respective channel inserts therein. Each channel insert may define a plurality of external grooves to form a plurality of microchannels when the channel inserts are received in the respective windows of the frame.

In particular, the channel inserts may provide a plurality of protrusions disposed along opposite walls of each channel insert, the grooves being provided between adjacent protrusions. In the assembled form of the channel assembly, the protrusions may abut opposite internal walls of each respective window of the frame such that microchannels are formed by an enclosed space between the grooves of the channel inserts and respective wall portions of the windows.

The frame and channel inserts may take any suitable shape and form. In one embodiment, the frame may have a general cylindrical hollow body and the plurality of windows may be disposed radially around a central axis of the cylindrical channel frame. Each window may be elongated in a direction parallel to the central axis of the frame. Similarly, each channel insert may have a corresponding elongate body for insertion into a respective frame window such that the protrusions of the channel inserts align and interface with internal walls of the respective frame windows to define the microchannels. In some embodiments, it may be desirable to provide a press fit between the channel inserts and the respective frame windows so as to provide a sealing interface between the internal walls of the frame windows and the protrusions of the respective channel inserts to define the microchannels therebetween.

The microchannels may be arranged in multiple layers, each layer including a radial array of microchannels.

Moreover, the microchannels may have any suitable orientation with respect to the channel member. For example, the channel member may have a central axis, and the microchannels may be oriented perpendicularly with respect to the central axis of the channel member. In some embodiments, the microchannels may be oriented at any suitable angle with respect to the central axis. For example, the microchannels May be oriented diagonally in the channel member. In some embodiments, the orientation of the microchannels may not be uniform, and the microchannels may extend at a combination of different angles with respect to the central axis of the channel member.

The microchannels can have any suitable length. Moreover, the microchannels may be generally of the same length or have different lengths.

In one embodiment, each of the microchannels are generally straight. In another embodiment, each of the microchannels are curved. In some embodiments, some microchannels may be straight and some microchannels may be curved.

In one embodiment, the passage control mechanism may include a barrier. The barrier may be movable between a first position in which the barrier prevents passage of micro-swimmers from the inlet sub-chamber to the outlet sub-chamber, and a second position in which passage of micro-swimmers from the inlet sub-chamber to the outlet sub-chamber via the microchannels are permitted.

The barrier may include a cover for covering one end of the microchannels in the first position. The cover may be removable from the microchannels in the second position. In particular, the barrier may be shaped to wrap around the channel member so as to block fluid communication from one end of the microchannels in the first position.

The barrier may be located at any suitable position relative to the microchannels and sub-chambers. In one embodiment, the barrier is located adjacent an inlet end of the microchannels in the first position, and the barrier is removed from the inlet end of the microchannels in the second position. The inlet end of the microchannels may be arranged for direct fluid communication with the inlet sub-chamber. Each of the microchannels may have an outlet end, arranged for direct fluid communication with the outlet sub-chamber.

In another embodiment, the passage control mechanism may include a first channel member defining a plurality of microchannels, and a second channel member defining a plurality of microchannels.

The two channel members may be movable relative to one another. In particular, the first and second channel members may be movable between a first position and a second position relative to one another such that in the first position, the microchannels in the first channel member are misaligned with the microchannels in the second channel member so as to prevent passage of micro-swimmers from the inlet sub-chamber to the outlet sub-chamber, and in the second position, the microchannels in the first channel member are at least partially aligned with the microchannels in the second channel member so as to permit passage of micro-swimmers from the inlet sub-chamber to the outlet sub-chamber via the microchannels.

The apparatus may further include an adjustment portion associated with the first channel member, and a pair of stoppers associated with the second channel member, wherein movement of the adjustment portion between the pair of stoppers moves the first and second channel members between the first and second positions. In some embodiments, the adjustment portion may be associated with the second channel member and the pair of stoppers may be associated with the first channel member without departing from the operating principle of the passage control mechanism. In particular, the adjustment portion may project from one channel member, and the stoppers may project from a corresponding end of the other channel member.

The two channel members can have any suitable regular or irregular shape. For example, the channel members may be generally cylindrical. More specifically, the channel members may have a hollow cylindrical body. The microchannels may be defined in the cylindrical wall of the hollow cylindrical body of each of the channel members.

In one embodiment, the first and second channel members may be concentric. The first channel member may surround the second channel member, or vice versa. Generally, when the first and second channel members move relative to one another, the microchannels in the two channel members moves in and out of alignment with one another.

In some embodiments, the passage control mechanism may include more than two channel members. For example, the passage control mechanism may include three, four or five channel members, without materially departing from the principle of operation of the passage control mechanism described herein. Each of the channel members may have a regular or irregular shape. In one embodiment, each channel member has a hollow cylindrical body, and each of the channel members are arranged concentrically with one another.

In one embodiment, the apparatus further includes a third channel member defining a plurality of microchannels. The first, second and third channel members may be movable relative to one another such that in the first position, the microchannels in at least two of the three channel members are misaligned to prevent passage of micro-swimmers from the inlet sub-chamber to the outlet sub-chamber, and in the second position, the microchannels in all three channel members are generally aligned to permit passage of micro-swimmers from the inlet sub-chamber to the outlet sub-chamber.

In one embodiment, the apparatus further includes a fourth channel member defining a plurality of microchannels, wherein the first, second, third and fourth channel members are movable relative to one another such that in the first position, the microchannels in at least two of the four channel members are misaligned to prevent passage of micro-swimmers from the inlet sub-chamber to the outlet sub-chamber, and in the second position, the microchannels in all four channel members are generally aligned to permit passage of micro-swimmers from the inlet sub-chamber to the outlet sub-chamber.

In one embodiment, the apparatus may further include a fifth channel member defining a plurality of microchannels, wherein the first, second, third, fourth and fifth channel members are movable relative to one another such that in the first position, the microchannels in at least two of the five channel members are misaligned to prevent passage of micro-swimmers from the inlet sub-chamber to the outlet sub-chamber, and in the second position, the microchannels in all five channel members are generally aligned to permit passage of micro-swimmers from the inlet sub-chamber to the outlet sub-chamber.

The apparatus may further include an inlet in fluid communication with the inlet sub-chamber, and an outlet in fluid communication with the outlet sub-chamber. In particular, the inlet may be in direct fluid communication with the inlet sub-chamber, and the outlet may be in direct fluid communication with the outlet sub-chamber.

The inlet sub-chamber may be arranged in any relative position with respect to the outlet sub-chamber. For example, the inlet sub-chamber may be spaced from the outlet sub-chamber, the inlet sub-chamber may be adjacent the outlet sub-chamber, the inlet sub-chamber may surround the outlet sub-chamber, or the outlet sub-chamber may surround the inlet sub-chamber.

In one embodiment, the inlet sub-chamber may occupy an outer part of the chamber, and the outlet sub-chamber may occupy an inner part of the chamber. In particular, the outlet sub-chamber may occupy a centre portion of the chamber, and the inlet sub-chamber may occupy an outer portion of the chamber adjacent a periphery of the chamber. In this embodiment, the inlet sub-chamber generally surrounds the outlet sub-chamber.

Each microchannel may have an inlet end adjacent the inlet sub-chamber and an outlet end adjacent the outlet sub-chamber such that micro-swimmers moving from the inlet sub-chamber to the outlet sub-chamber passes through the microchannels from the respective inlet ends to the outlet ends. A distance between the inlet ends of the microchannels and an inner peripheral surface of the chamber may define a thickness of the outlet sub-chamber. The outlet sub-chamber may have any suitable thickness. For example, the thickness of the outlet sub-chamber may be generally between 0.2 mm to 6 mm. In some embodiments, the thickness of the sub-chamber may be generally between 0.5 mm to 2 mm.

The apparatus may further include a transfer member movable within the outlet sub-chamber for transferring micro-swimmers from the outlet sub-chamber to the outlet. The transfer member may be of any suitable shape of form. For example, the transfer member may have a generally cylindrical body. The cylindrical body of the transfer member may be slidable from a first end of the outlet sub-chamber to an opposite second end of the outlet sub-chamber to transfer the micro-swimmers to the outlet. The apparatus may further include a cap for the outlet.

In some embodiments, the transfer member may be used to load the chamber with a buffer fluid and/or a sample of micro-swimmers. In particular, movement of the transfer member from the second end of the outlet sub-chamber to the first end of the outlet sub-chamber may be used to draw the buffer fluid and/or a sample of micro-swimmers into the chamber.

The apparatus may include any suitable number of microchannels. For example, the total number of microchannels may be between 60 and 70,000. In some embodiments, the total number of microchannels may be between 500 and 20,000.

The cross-sectional area of each microchannel may be generally between 1,500 μm² to 5,000,000 μm².

Any suitable manufacturing process may be used to manufacture the apparatus. In one embodiment, the apparatus may be manufactured via a process including injection moulding.

According to another aspect of the invention, there is provided a method of separating micro-swimmers using the apparatus as described herein. The method may include the steps of
loading a buffer fluid into the chamber,
configuring the passage control mechanism in the first condition,
loading a sample of micro-swimmers into the inlet sub-chamber,
configuring the passage control mechanism in the second condition, and
collecting the micro-swimmers from the outlet sub-chamber.

After the step of configuring the passage control mechanism in the second condition, the method may include the step of waiting until the end a predetermined time period prior to collecting the micro-swimmers from the outlet sub-chamber. The predetermined time period may be generally between 10 minutes and 90 minutes. More particularly, the predetermined time period may be roughly 10 to 20 minutes.

After the step of waiting, the method may further include the step of configuring the passage control mechanism in the first condition prior to collecting the micro-swimmers from the outlet sub-chamber.

The step of collecting may include transferring the micro-swimmers from the outlet sub-chamber to an outlet using a transfer member, wherein the transfer member is movable within the outlet sub-chamber to transfer the micro-swimmers.

According to another aspect of the invention, there is provided a method of separating micro-swimmers, the method including the steps of
loading a buffer fluid into a reservoir, the reservoir having an inlet reservoir and an outlet reservoir,
sealing the inlet reservoir,
loading a sample of micro-swimmers into the inlet reservoir,
directing passage of micro-swimmers from the inlet reservoir to the outlet reservoir via a plurality of microchannels, and
collecting the micro-swimmers from the outlet reservoir.

After the step of directing, the method may include the step of waiting until the end a predetermined time period prior to collecting the micro-swimmers from the outlet reservoir. The predetermined time period may be generally between 10 minutes and 90 minutes. More particularly, the predetermined time period may be roughly between 10 to 20 minutes.

After the step of waiting, the method may further include the step of sealing the outlet reservoir prior to collecting the micro-swimmers from the outlet sub-chamber.

The step of collecting may include transferring the micro-swimmers from the outlet reservoir to an outlet using a transfer member, wherein the transfer member is movable within the outlet reservoir to transfer the micro-swimmers.

In order that the invention may be more readily understood and put into practice, one or more preferred embodiments thereof will now be described, by way of example only, with reference to the accompanying drawings.

### BRIEF DESCRIPTION OF DRAWINGS

FIG. 1A is a perspective view of an apparatus for separating micro-swimmers according to one embodiment of the invention.

FIG. 1B is an exploded assembly view of the apparatus shown in FIG. 1A.

FIG. 1C is a sectional view of the chamber, channel member and outlet of the apparatus shown in FIGS. 1A and 1B.

FIG. 1D is a close-up view of the microchannels in a portion of the channel member shown in FIG. 1C.

FIG. 2A is a perspective view illustrating a passage control mechanism according to an alternative embodiment of the invention.

FIG. 2B is an exploded assembly view of the passage control mechanism shown in FIG. 2A.

FIG. 2C is an elevated view of the passage control mechanism shown in FIGS. 2A and 2B arranged in a passage position to allow passage of micro-swimmers through the microchannels.

FIG. 2D is an elevated view of the passage control mechanism shown in FIGS. 2A and 2B arranged in a non-passage position to prevent passage of micro-swimmers through the microchannels.

FIG. 3A is a partial perspective view of a passage control mechanism according to another embodiment of the invention.

FIG. 3B is a partial perspective view of the passage control mechanism shown in FIG. 3A arranged in a non-passage position to prevent passage of micro-swimmers through the microchannels.

FIG. 3C is a partial perspective view of the passage control mechanism shown in FIG. 3A arranged in a passage position to allow passage of micro-swimmers through the microchannels.

FIG. 4A is a partial perspective view of a channel member defining bent microchannels according to an embodiment of the invention.

FIG. 4B is an elevated view of the apparatus including a channel member defining curved microchannels according to an embodiment of the invention.

FIGS. 5A to 5D are schematic diagrams illustrating a method of using the apparatus shown in FIGS. 1A to 1C to separate micro-swimmers according to one embodiment of the invention.

FIG. 6A is a graph illustrating concentration of sperm collected from the outlet of the experimental apparatus after an incubation time period of 10 mins, 15 mins, 20 mins compared to the initial sperm sample using bull sperm.

FIG. 6B is a graph illustrating the percentage of recovered sperm collected from the outlet of the experimental apparatus after an incubation time period of 10 mins, 15 mins, 20 mins using bull sperm in comparison with estimations generated by a computational model. The bar graph indicates experimental results and the dashed line indicates the trend for the percentage of recovered sperm (A) estimated by a computational model.

FIG. 6C is a graph illustrating the retrieval efficiency of healthy sperm collected from the outlet of the experimental apparatus after an incubation time period of 10 mins, 15 mins, 20 mins.

FIG. 7A is a graph illustrating mean head width (with standard deviation from at least three independent experiments) of the separated sperm using the experimental apparatus for sperm morphology and DNA integrity analysis after an incubation time period of 10 mins, 15 mins, 20 mins as compared with the initial semen sample using bull sperm.

FIG. 7B is a graph illustrating mean head length (with standard deviation from at least three independent experiments) of the separated sperm using the experimental apparatus for sperm morphology and DNA integrity analysis after an incubation time period of 10 mins, 15 mins, 20 mins as compared with the initial semen sample using bull sperm.

FIG. 7C is a graph illustrating mean percentage of DNA fragmentation index (% DFI) (with standard deviation from at least three independent experiments) of the separated sperm using the experimental apparatus for sperm morphology and DNA integrity analysis after an incubation time period of 10 mins, 15 mins, 20 mins as compared with the initial semen sample using bull sperm.

FIG. 8A is a graph illustrating mean sperm concentration (with standard deviation from at least five independent experiments) for the initial semen sample and the separated sperm using the experimental apparatus after an incubation period of 15 mins for selection throughput and quality analysis using human sperm.

FIG. 8B is a graph illustrating mean % DFI (with standard deviation from at least five independent experiments) for the initial semen sample and the separated sperm using the experimental apparatus after an incubation period of 15 mins for selection throughput and quality analysis using human sperm.

FIG. 8C is a graph illustrating mean percentage of sperm with normal morphology (with standard deviation from at least five independent experiments) for the initial semen sample and the separated sperm using the experimental apparatus after an incubation period of 15 mins for selection throughput and quality analysis using human sperm.

FIGS. 9A and 9B are partial assembly views of a channel member according to another embodiment.

FIG. 9C is a perspective view of the fully assembled channel member of FIGS. 9A and 9B.

FIG. 9D is a cross sectional view of the assembled channel member of FIG. 9C.

FIG. 10A is a graph illustrating mean sperm concentration for the initial semen sample and the separated sperm using the experimental apparatus having a channel assembly as shown in FIGS. 9A to 9D after an incubation period of 15 mins for selection throughput and quality analysis using bull sperm.

FIG. 10B is a graph illustrating mean sperm vitality for the initial semen sample and the separated sperm using the experimental apparatus having a channel assembly as shown in FIGS. 9A to 9D after an incubation period of 15 mins for selection throughput and quality analysis using bull sperm.

### DETAILED DESCRIPTION

Whilst the following embodiments will be described with reference to separation of sperm, as previously mentioned, sperm separation may include any one or more of: selection, purification, rapid selection, and high throughput selection of motile cells.

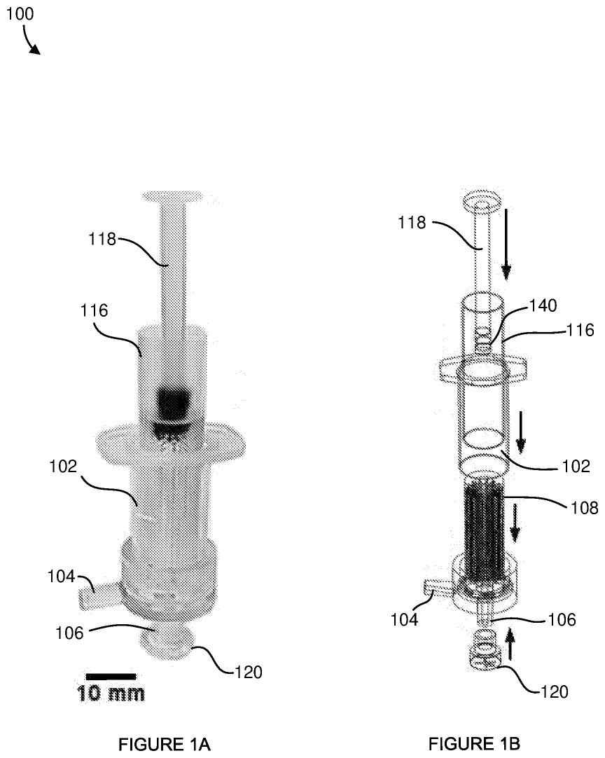

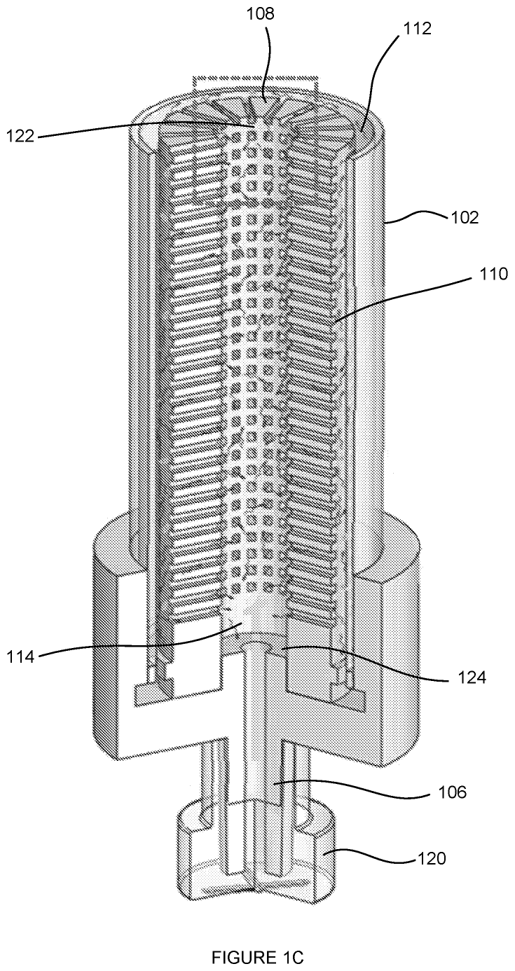

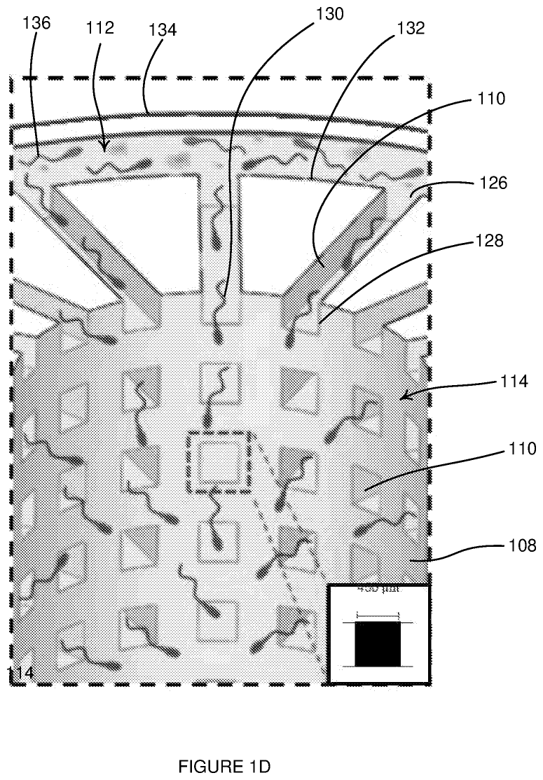

As shown in FIGS. 1A and 1B, the apparatus 100 for separating sperm includes a main chamber 102 having an inlet 104 for receiving a sample of sperm and an outlet 106 for transferring the separated sperm. As more clearly shown in FIG. 1C, the main chamber 102 holds a channel member 108 defining a plurality of microchannels 110. The microchannels 110 are distributed across the three-dimensional (3D) hollow cylindrical body of the channel member 108. In particular, the microchannels 110 are arranged in a plurality of layers stacked on top of one another. Each layer provides a radial array of microchannels 110 extending outwardly from the bore the hollow cylindrical body of the channel member 108 to an outer surface 132 of the channel member 108.

The channel member 108 within the chamber 102 divides the chamber into different sections. In particular, the main chamber 102 is divided into an inlet sub-chamber 112 for direct fluid communication with the inlet 104, and an outlet sub-chamber 114 in direct fluid communication with the outlet 114. The plurality of microchannels 110 are disposed between the inlet sub-chamber 112 and the outlet sub-chamber 114.

In the embodiment shown in FIG. 1C, the inlet sub-chamber 112 occupies an outermost portion of the main chamber 102, adjacent a periphery of the main chamber 102. In particular, the inlet sub-chamber 112 is defined by a volume of space between an inner surface 134 of the periphery of the main chamber 102 and an outer surface 132 of the channel member 108. The outlet sub-chamber 114 occupies a central portion of the main chamber 102. The outlet sub-chamber 114 is defined by a volume of space within the bore of the hollow cylindrical channel member 108. The channel member 108 surrounds the outlet sub-chamber 114, and the inlet sub-chamber 112 surrounds the channel member 108.

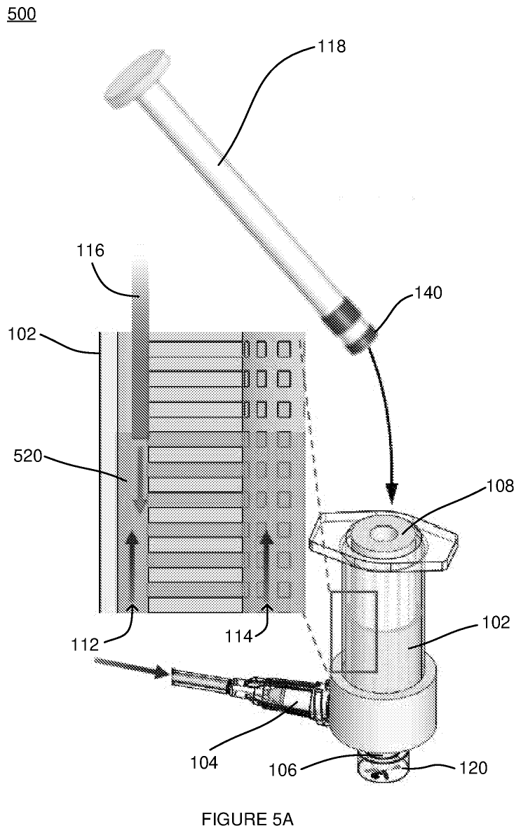

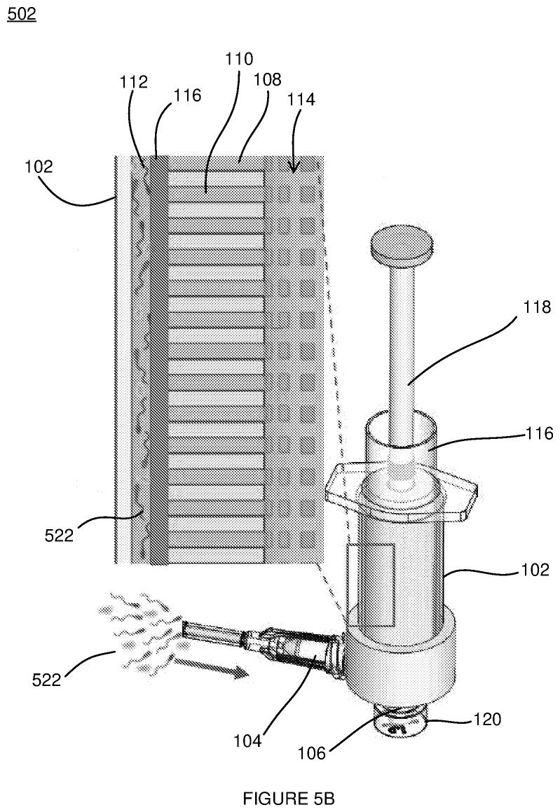

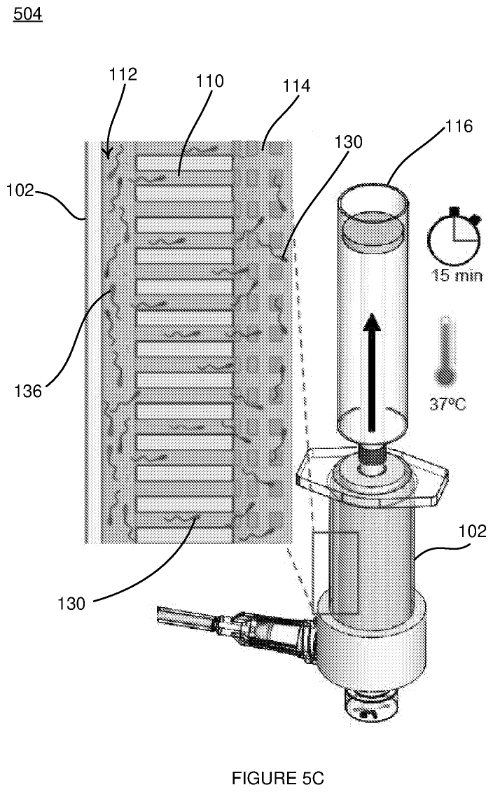

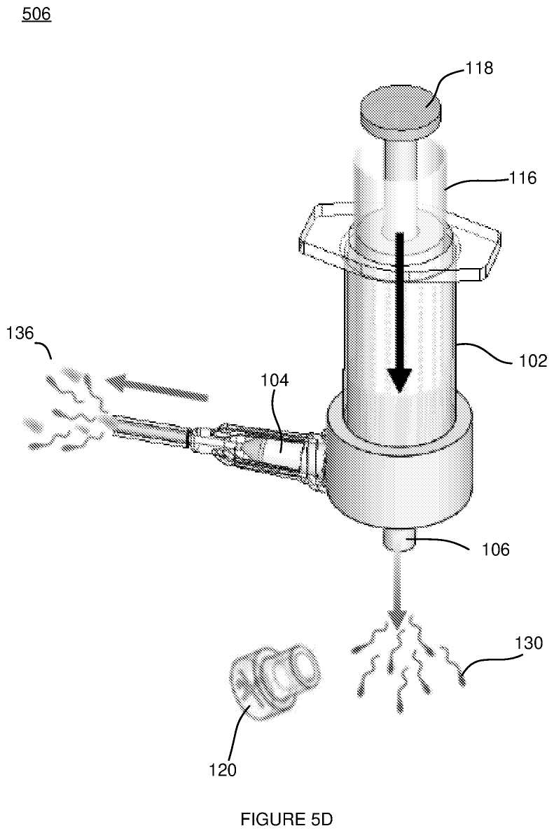

As more clearly shown in FIG. 1D, each microchannel 110 has an inlet end 126 adjacent the inlet sub-chamber 112 and an outlet end 128 adjacent the outlet sub-chamber 114. During use, motile sperm 130 from the inlet sub-chamber 112 passes through the microchannels 110 from the respective inlet ends 126 to the outlet ends 128 and enters the outlet sub-chamber 114. Meanwhile, non-motile sperm 136 remains in the inlet sub-chamber 112. A process of using the apparatus 100 will be described in more detail below with reference to FIGS. 5A to 5D.

The channel member 108 provides a 3D network of microchannels 110 that replicates the highly parallelized and 3D selection process in vivo that enables a single-cell level sorting mechanism within a relatively short timeframe. Specifically, the 3D folded structure of the reproductive tract breaks down the initial sample volume (˜1-4 ml) into a few microliters per selection event (i.e. folded epithelial tissue) to process over hundreds of millions of sperm in just a few hours.

The apparatus 100 thereby essentially provides a 3D sorting platform for high-throughput sperm selection based on their boundary-following behaviour through a 3D network of microchannels 110.

Generally, the channel member 108 may provide a total number of 60 to 70,000 microchannels 110. In some embodiments, the channel member 108 may provide a total number of 500 to 20,000 microchannels. The cross-sectional area of each microchannel may be generally between 1,500 μm2 to 5,000,000 μm2.

A distance between the outer surface 132 of the channel member 108 and the inner peripheral surface 134 of the chamber 102 defines a thickness of the inlet sub-chamber 112. In some embodiments, the thickness of the inlet sub-chamber 112 may be generally between 0.2 mm to 6 mm. In some embodiments, the thickness of the inlet sub-chamber 112 may be generally between 0.5 mm to 2 mm. In some embodiments, a reduction of thickness of the inlet sub-chamber 112 may provide a more effective volume to allow better interface between the initial semen sample and the microchannels 110.

The apparatus 100 further includes a passage control mechanism 116 in the form of a moveable barrier for controlling the passage of sperm from the inlet sub-chamber 112 to the outlet sub-chamber 114 via the microchannels 110. The barrier 116 is moveable in and out of the chamber 102. In particular, the barrier 116 can be moved to a non-passage condition in which the barrier 116 is positioned within the chamber 102 to cover the outer surface 132 of the channel member 108. When the channel member 108 is covered by the barrier 116, an inlet end 126 (see FIG. 1D) of the microchannels 110 is blocked and as such passage of sperm from the inlet sub-chamber 112 to the outlet sub-chamber 114 via the microchannels 110 can be prevented. The barrier 116 can also be moved to a passage condition in which the barrier 116 is removed from the chamber 102. In this passage condition, the channel member 108 is no longer covered by the barrier 116 and passage of sperm from the inlet sub-chamber 112 to the outlet sub-chamber 114 via the microchannels 110 are permitted.

The apparatus 100 further includes a transfer member 118 movable within the outlet sub-chamber 114 for transferring the sperm from the outlet sub-chamber 114 to the outlet 106. The transfer member 118 shown in FIGS. 1A and 1B has a generally cylindrical body which is slidable within the outlet sub-chamber 114. An end portion 140 of the transfer member 120 is designed to correspond with the bore dimensions of the hollow cylindrical channel member 108 (which are also the dimensions of the outlet sub-chamber 114). During use, the end portion 140 of the transfer member 118 is slidable from a first end 122 of the outlet sub-chamber 114 to an opposite second end 124 of the outlet sub-chamber 114 to transfer the sperm in the outlet sub-chamber 114 to the outlet 106. The apparatus also includes a cap 120 for the outlet 106.

In some embodiments, the transfer member 118 can also be used to load the chamber 102 with a buffer fluid and/or a sample of sperm. The transfer member 118 may operate in a similar manner to a syringe plunger. In particular, movement of the transfer member 118 from the second end 124 of the outlet sub-chamber 114 to the first end 122 of the outlet sub-chamber 114 while the cap 120 is placed on the outlet 106 may be used to draw the buffer fluid and/or a sample of sample into the chamber 102 from the inlet 104.

Alternatively, the buffer fluid can also be loaded from the outlet 106 whilst a cap (not shown) is placed over the inlet 104. In particular, movement of the transfer member 118 from the second end 124 of the outlet sub-chamber 114 to the first end 122 of the outlet sub-chamber 114 while a cap is placed on the inlet 104 may be used to draw the buffer fluid and/or a sample of sample into the chamber 102 from the outlet 106.

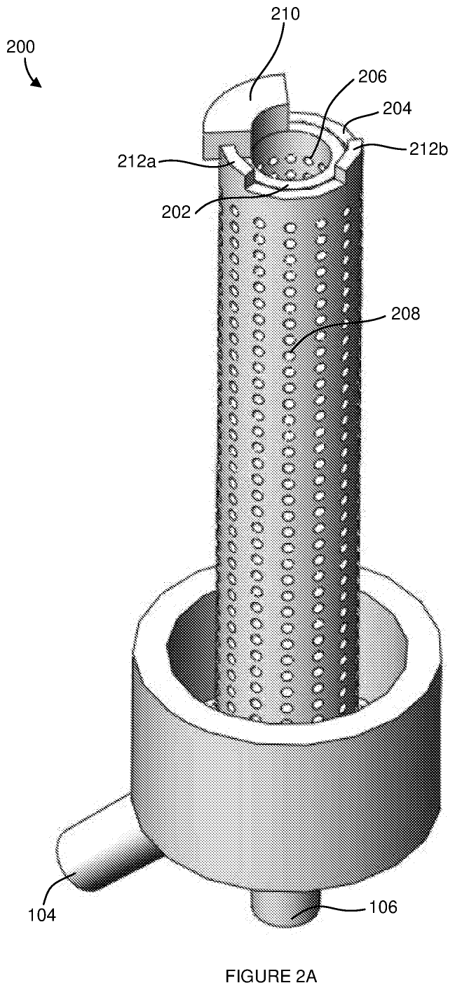

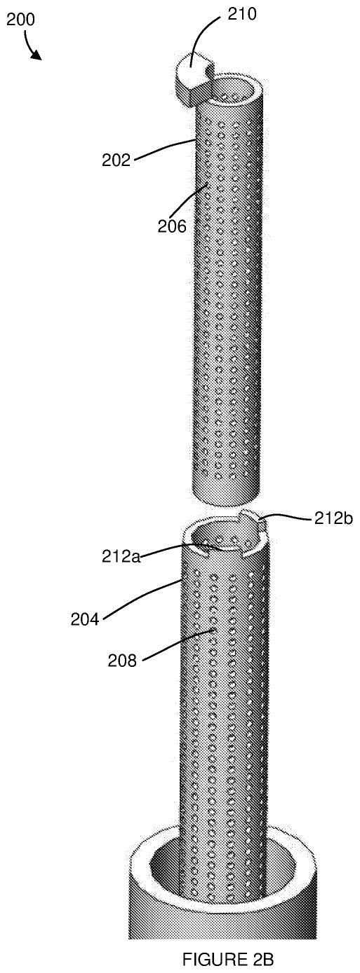

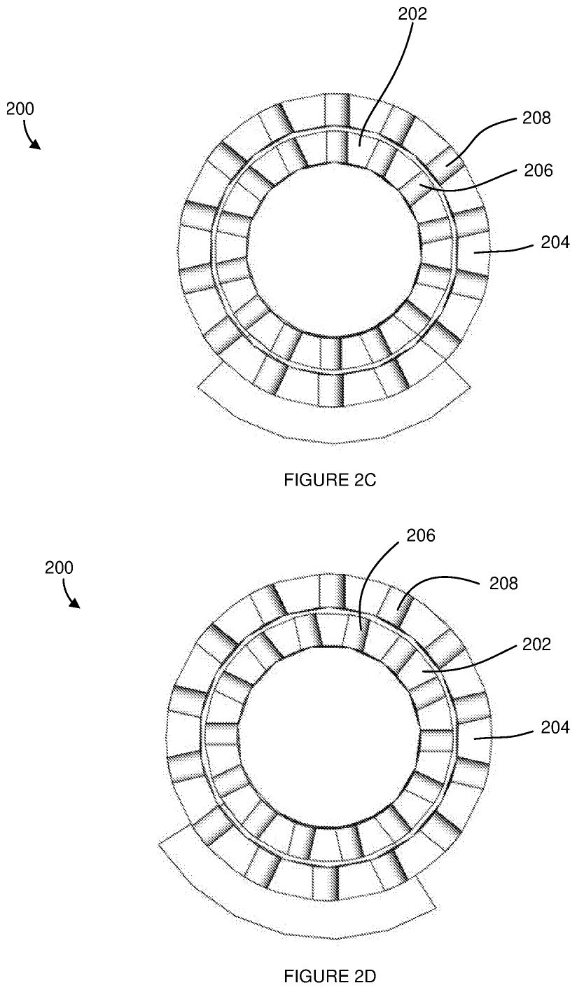

FIGS. 2A to 2D illustrate an alternative passage control mechanism 200 according to another embodiment of the invention. The passage control mechanism 200 includes an inner channel member 202 and an outer channel member 204, each of the two channel members 202, 204 defining a plurality of microchannels 206, 208.

Each of the two channel members 202, 204 have a hollow cylindrical body, the inner channel member 202 being configured to fit inside the outer channel member 204 such that the two channel members 202, 204 are concentric. In particular, an external diameter of the inner channel member 202 corresponds with an internal diameter of the outer channel member 204 such that an outer surface of the inner channel member 202 contacts an inner surface of the outer channel member 204.

As more clearly shown in FIGS. 2C and 2D, the two channel members are rotatable relative to one another. In particular, the inner channel member 202 may be rotatable relative to the outer channel member 204 into a passage position as shown in FIG. 2C and a non-passage position as shown in FIG. 2D. In the passage position illustrated in FIG. 2C, the microchannels 206 of the inner channel member 202 are aligned with the microchannels 208 of the outer channel member 204 so as to permit passage of sperm through the microchannels 206, 208 of the passage control mechanism 200. In the non-passage position illustrated in FIG. 2D, the microchannels 206 of the inner channel member 202 are misaligned with the microchannels 208 of the outer channel member 204 so as to prevent passage of sperm through the microchannels 206, 208 of the passage control mechanism 200.

Now referring back to FIGS. 2A and 2B, an adjustment portion 210 is provided at an end of the inner channel member 202, and a pair of stopper members 212a, 212b is provided at a corresponding end of the outer channel member 204. Movement of the adjustment portion 210 between the pair of stopper members 212a, 212b rotates the inner channel member 202 relative to the outer channel member 204 between the passage and non-passage positions shown in FIGS. 2C and 2D.

In the embodiment shown in FIGS. 2A to 2B, the channel members 202, 204 providing the microchannels 206, 208 and passage control mechanism 200 are incorporated into the same components so that a separate barrier for insertion into, and removal from, the chamber 102 is not required. Whilst not shown in FIGS. 2A and 2B, it will be understood that the passage control mechanism 200 is mounted inside a main chamber 102 such that the barrier control mechanism 200 divides the space within the main chamber 102 into an inlet sub-chamber 112 and an outlet chamber 114. The inlet chamber 112 being outside the outer channel member 204 and the outlet sub-chamber 114 being inside the inner channel member 202, analogous to the apparatus 100 shown in FIGS. 1A and 1B.

During use, when the inlet sub-chamber 112 is being loaded with a sample of sperm, the passage control mechanism 200 is moved into the non-passage position (FIG. 2D) to prevent passage of sperm from the inlet sub-chamber 112 to the outlet sub-chamber 114. Once the sperm sample is fully loaded, the passage control mechanism 200 can be moved into the passage position (FIG. 2C) to permit passage of motile sperm 130 from the inlet sub-chamber 112 to the outlet sub-chamber 114 via the aligned microchannels 206, 208.

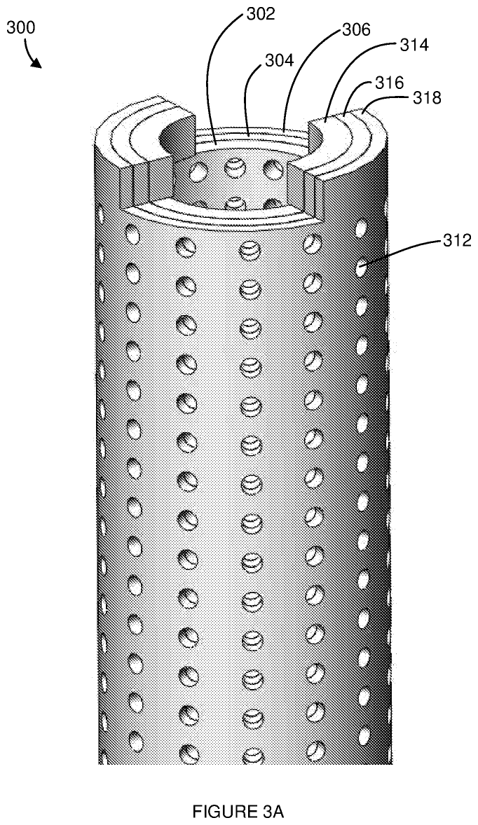

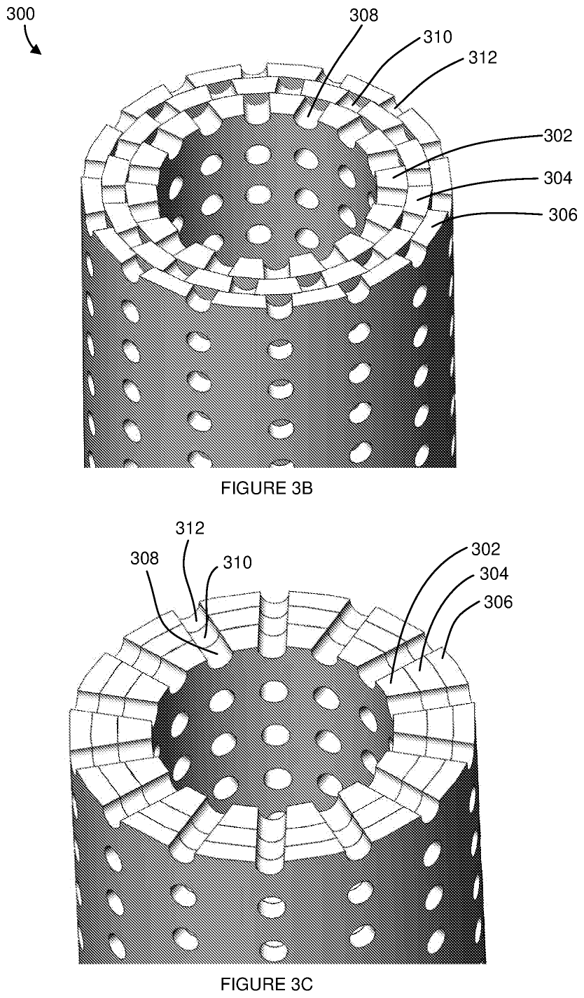

As shown in FIGS. 3A to 3C, an alternative passage control mechanism 300 according to a further embodiment of the invention includes three concentric channel members 302, 304, 306, each of the three channel members 302, 304, 306 defining a plurality of microchannels 308, 310, 312 therein.

The passage control mechanism 300 operates based on the same operating principal as the passage control mechanism 200 previous described. In particular, relative rotation between the three channel members 302, 304, 306 move the respective microchannels 308, 310, 312 in and out of alignment with one another to either allow or prevent passage of sperm via the microchannels 308, 310, 312. As shown in FIG. 3B, the channel members 302, 304, 306 are rotated into a non-passage position in which the microchannels 308, 310, 312 provided by the respective channel members 302, 304, 306 are misaligned to prevent passage of sperm through the microchannels 308, 310, 312. As shown in FIG. 3C, the channel members 302, 304, 306 are rotated into a passage position in which the microchannels 308, 310, 312 provided by the respective channel members 302, 304, 306 are aligned to allow passage of sperm through the microchannels 308, 310, 312.

Now referring back to FIG. 3A, adjustment portions 314, 316, 318 projecting from one end of the respective channel members 302, 304, 306 can be used to control relative rotational movements of the channel members 302, 304, 306. In particular, the adjustment portions 314, 316, 318 can be used as alignment guides such that alignment of the adjustment portions 314, 316, 318 indicates alignment of the microchannels 308, 310, 312 as more clearly shown in FIG. 3C. Conversely, any misalignment of the adjustment portions 314, 316, 318 would indicate corresponding misalignment in the microchannels 302, 304, 306.

In some embodiments, alternative passage control mechanisms may include more concentric channel members. For example, some passage control mechanism may include four or five concentric channel members, without materially departing from the operating principle of the passage control mechanisms 200, 300 described herein. An increased number of concentric channel members may provide more effective blockage of motile sperm movement through the microchannels, when the microchannels are misaligned in the non-passage position.

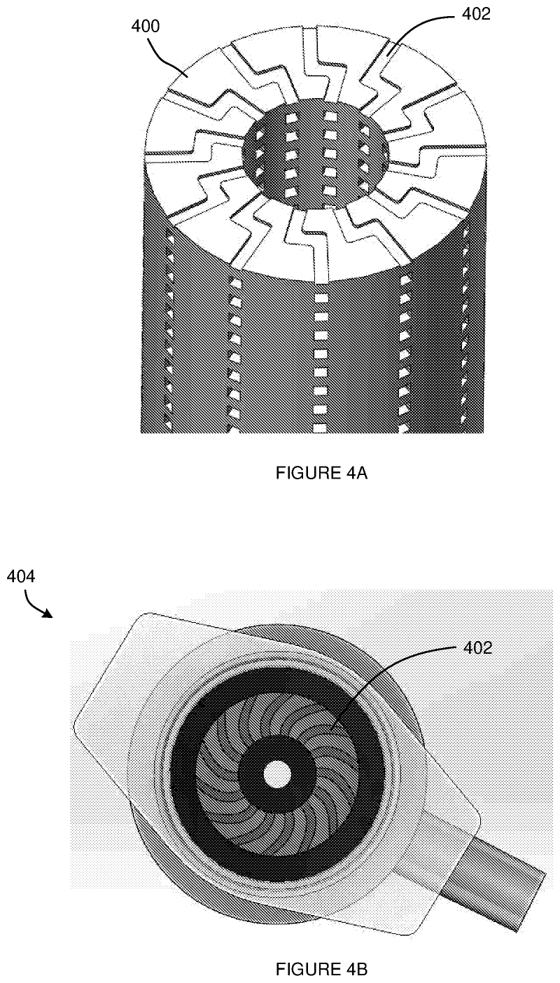

FIGS. 4A and 4B illustrate an apparatus 404 according to further embodiments of the invention in which a channel member 400 defines bent or curved microchannels 402, 406. In particular, FIG. 4A illustrates a partial channel member 400 defining bent microchannels 402. FIG. 4B illustrates an apparatus 404 in which the channel member defines curved microchannels 406. An apparatus with bent or curved microchannels 402, 406 would operate generally in the same manner as apparatus 100 as described herein. In some embodiments, the bent or curved microchannels 402, 406 may more closely mimic the geometry of the folded epithelial groove in the fallopian tube.

A method of separating sperm using the apparatus 100 as shown in FIGS. 1A to 1C will now be described with reference to FIGS. 5A to 5D.

FIG. 5A illustrates that at an initial method step 500, the transfer member 118 and barrier 116 are removed from the chamber 102, and the cap 120 is placed over the outlet 106 to provide a stagnant flow environment in the chamber 102. The chamber 102 is then loaded with a buffer fluid 520 via the inlet 104. As the chamber 102 is prefilled with the buffer fluid 520, the buffer fluid enters the inlet sub-chamber 112, the microchannels 110 and the outlet sub-chamber 114. In embodiments of the apparatus incorporating a passage control mechanism 200, 300 such as those shown in FIGS. 2A to 3C, the concentric channel members of the respective passage control mechanism 200, 300 would be rotated into the passage position prior to loading the buffer fluid 520 so that the buffer fluid 520 can enter the microchannels, inlet and outlet sub-chambers 112, 114.

Once loading of the buffer fluid is complete, the barrier 116 is inserted into the chamber 102 to cover the inlet ends 126 of the microchannels 110, thereby effectively blocking entry into the microchannels 110 from the inlet sub-chamber 112. For passage control mechanisms 200, 300, the respective channel members would be rotated into the non-passage position after the loading of the buffer fluid 520 is complete.

As shown in FIG. 5B, at method step 502, once the barrier 116 is fully inserted into the chamber 102, a sample of semen 522 is inserted into the inlet sub-chamber 112 via the inlet 104, for example, using a syringe. The semen sample 522 mixes with the buffer fluid 520 in the inlet sub-chamber 112 but is prevented from entering the microchannels 110 of the channel member 108 by the barrier 116 (or the misalignment of the channel members in the alternative passage control mechanism 200, 300).

Now referring to FIG. 5C, at method step 504, the barrier 116 is removed from the chamber 102 and apparatus 100 containing the buffer fluid 520 and semen sample 522 is incubated for a predetermined time period at a predetermined temperature. In embodiments incorporating alternative passage control mechanism 200, 300, the respective channel members are moved into the passage position prior to the start of the predetermined incubation time period.

In one embodiment, the predetermined incubation temperature may be 37° C. In some embodiments, the predetermined incubation time period may be generally between 10 minutes and 90 minutes. More particularly, the predetermined time period may be roughly 10 to 20 minutes.

During incubation, motile sperm 130 can swim through the microchannels 110 of the channel member 108 to enter the outlet sub-chamber 114, leaving non-motile sperm 136 behind in the inlet sub-chamber 112.

In method step 506 as illustrated in FIG. 5D, after expiry of the predetermined incubation time period, the barrier 116 is re-inserted into the chamber 102 effectively separating the non-motile sperm 136 residing in the inlet sub-chamber 112 from the motile sperm 136 in the microchannels 110 and the outlet sub-chamber 114. In embodiments incorporating alternative passage control mechanisms 200, 300, the channel members are moved back to the non-passage position to provide the same separation.

Once the separation is in place, non-motile sperm 136 is removed from the inlet sub-chamber 112 via the inlet 104. Then, the outlet cap 120 is removed, and the transfer member 118 is moved from one end of the outlet sub-chamber 114 to an opposite end of the outlet sub-chamber 114 to transfer the motile sperm 130 from the outlet sub-chamber 114 to the outlet 130 for collection.

Selection of high-quality sperm is crucial to assisted reproduction. However, conventional clinical methods for sperm selection are highly manual, time-consuming, and prone to operator errors. Embodiments of the apparatus for separating sperm as described herein is simple in operation, scalable, and clinically applicable. As mentioned, embodiments of the apparatus mimic the highly parallelised 3D sperm selection process in vivo. In particular, the 3D network of microchannels is capable of facilitation selection of high-quality sperm based on their boundary following behaviour.

According to experimental results, an apparatus 100 with a channel member 108 providing 560 parallel microchannels 110 is capable of selecting up to 42% of healthy motile sperm 130 directly from the raw semen sample 522 in under 15 minutes. This improvement in selection throughput is significant, outperforming previously developed microfluidic technologies by at least 38% to provide a sufficient volume (˜500 μL) and number (1,750,000) of high-quality sperm for droplet-based IVF and IUI.

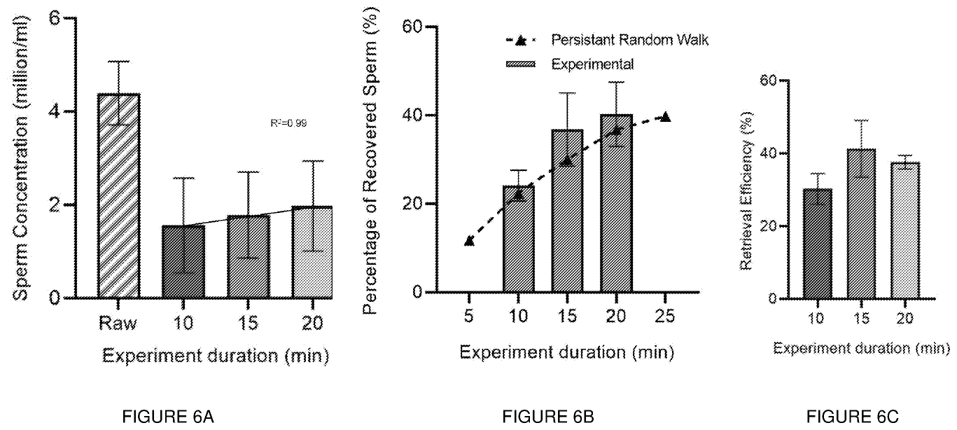

As described below with reference to FIG. 6A to 8C, the performance of the apparatus 100 is evaluated through experiments with bull and human sperm. The experimental results demonstrate performance capabilities of the apparatus 100 to select sperm with 73% improvement in both morphology and DNA integrity, considerably outperforming the current best clinical practices. This approach enables the selection of a subpopulation of high-quality human sperm from an oligozoospermia sample with a DNA integrity below the reference value for fertility. The fabrication method of the apparatus is also simple and scalable, representing a commercially viable technology for translation and clinical adoption. The apparatus provides a promising opportunity to more frequently perform IUI and IVF in fertility clinics, instead of ICSI, reducing the associated clinical workload and invasive steps to improve fertility care worldwide.

### Experimental Results and Discussion

Experiments were carried out using an apparatus 100 as shown in FIGS. 1A to 1D (hereinafter referred to as the ‘experimental apparatus’). The channel member 108 of the experimental apparatus provided a network of 560 parallel microchannels 110, and a main chamber 102 size of 5 mL. The experimental apparatus provided a maximum contact area between 1.5 mL of raw semen sample and selection events (˜13 μL of sample per μm² of selection area) to enable a highly parallelized and rapid sorting mechanism.

The inlet sub-chamber 112 provided a volume of 1.5 mL. The channel member 108 included a network of 450 μm×450 μm microchannels in a hollow cylindrical body (35 layers of 16 radial microchannels). The outlet sub-chamber had a volume of 500 μL. This geometry, interfaces 13 μL of raw semen per μm² area of each microchannel to facilitate sperm entrance into the microchannels 110.

Due to this considerably high surface to volume ratio, motile sperm can easily find and enter the microchannels 110 based on their swimming behaviour while non-motile sperm, somatic cells and debris remain in the inlet sub-chamber 112 (FIGS. 1C and 1D). The geometry of the experimental apparatus 100 was optimised based on several key considerations to ensure maximum throughput, including (i) the geometry of the folded epithelial groove in the female fallopian tube that guides sperm, (ii) space restrictions to maximize the contact area between the raw sample and the clean buffer, (iii) the required selection length to ensure the highest quality of selected sperm, and (iv) the required sample volume for performing an IUI procedure.

The apparatus 100 is readily translatable using mass manufacturing methods such as plastic injection moulding to more readily meet the regulatory requirements for a medical device. Implementing mass manufacturing techniques even provide an additional opportunity to improve the throughput of the apparatus 100 by at least 16-folds, as an improvement in the printing resolution from 400 μm to ˜100 μm can result in an additional selection 8800 microchannels.

The operational procedure of experimental apparatus 100 is simple and adoptable to the clinical workflow (FIGS. 5A to 5D). Prior to the experiment, the chamber 102 is prefilled with a buffer fluid by applying a negative pressure and after incubation at 37° C. for ˜30 minutes to reach the physiological temperature, the experimental apparatus was tested with frozen bull and fresh human semen samples. To characterize the performance of the experimental apparatus for oligozoospermia, the experimental apparatus was tested with diluted samples that represent a concentration lower than the reference limit of 15 million sperm per ml according to the World Health Organisation (WHO) guideline. Upon loading 1.5 ml of semen sample (a clinically relevant volume) into the inlet sub-chamber (FIG. 5B), motile sperm swim through the microchannels due to their boundary-following behaviour to reach the outlet sub-chamber while non-motile sperm, somatic cells and debris remain in the inlet sub-chamber (FIG. 5C). At the end of experiment duration, 500 μL of buffer containing selected sperm is retrieved from the outlet sub-chamber 114 (FIG. 5D), a clinically relevant volume for application in not only droplet-based IVF but also IUI. It is noteworthy that, to prevent flow and mixing, the experimental apparatus 100 was designed to ensure the same hydrostatic pressure distribution in the inlet and outlet sub-chambers 112, 114 during operation. Moreover, a rolled laminate sheet was used as the barrier 116 to eliminate the flow and prevent mixing when loading the semen sample and unloading the selected sperm.

### Apparatus Selection Throughput

FIGS. 6A to 6C illustrate the selection throughput of the experimental apparatus as a function of time. To investigate the effect of experiment duration (also referred to herein as the ‘incubation time period’) on selected sperm concentration and recovery rate, the experimental apparatus 100 was tested with frozen bull sperm for 10, 15, and 20 minutes. FIG. 6A shows the selected sperm concentration from the experimental apparatus in comparison with the concentration of the initial semen sample 522 loaded on-chip. Using samples with an average concentration of 4.4 million per mL in the inlet sub-chamber 112, selected sperm concentration increased linearly (R2=0.99) with time from 1.6 million per mL to 1.9 million per mL by increasing the experiment duration from 10 to 20 minutes. Specifically, the experimental apparatus 100 selects over 800,000 motile sperm out of the total of 6,600,000 sperm (motile, immotile, and dead) introduced in the inlet sub-chamber 112.

Based on this selection throughput, the percentage of recovered sperm (the number of selected sperm expressed as a percentage of the total number of sperm in the initial semen sample) was 24%±3%, 37%±8%, and 40%±7% for 10-minute, 15-minute, and 20-minute experiments, respectively (FIG. 6B). The observed increase in collected sperm number is due to the longer selection time that allows the less motile sperm to reach the outlet sub-chamber 114.

To compare the experimental results with theory, bull sperm motility in the experimental apparatus 100 was modelled as a persistent random walk (PRW) and only Brownian motion was considered for non-motile sperm (FIG. 6B).

Simulations predicted almost the same percentage of recovered after 10 minutes (22.4%), and lower percentages of recovered sperm for 15 minutes and 20 minutes experiments (30% and 37% respectively) as compared with the experimental measurements (FIG. 6B). The simulation results indicate that the recovery rate will saturate by increasing the selection time to over 25 minutes (the number of sperm reaching the outlet sub-chamber is the same as the number of sperm leaving it).

To evaluate the performance of the experimental apparatus 100 for selecting live sperm, both the initial and selected sperm from the experimental apparatus 100 were stained after the selection process using live/dead viability kit. FIG. 6C shows the retrieval efficiency of healthy sperm (percentage of live sperm 130 retrieved from initial raw sample 522) for 10, 15 and 20 minute experiments. The retrieval efficiency of healthy sperm increased with experiment duration from 30%±4% at 10 minutes to 41±7% at 15 minutes; however, decreased to 38%±2% by further increasing the incubation time to 20 minutes. The results indicate that, for experiment duration of 20 min, a subpopulation of slower sperm can reach the selection chamber (compare to a 15 minute experiment duration) but they also die faster, potentially due to the exhaustion of sperm and long exposure to the remaining uncured resin in the experimental apparatus 100. Taken together, the experimental apparatus 100 provides a high-throughput approach for sperm selection, considerably outperforming conventional clinical practices and previously developed microfluidic methods. The experimental apparatus 100 selects over 800,000 sperm in 500 μL that can be applied directly for IUI and droplet-based IVF, or for sperm selection prior to ICSI.

### Selected Sperm Morphology and DNA Integrity

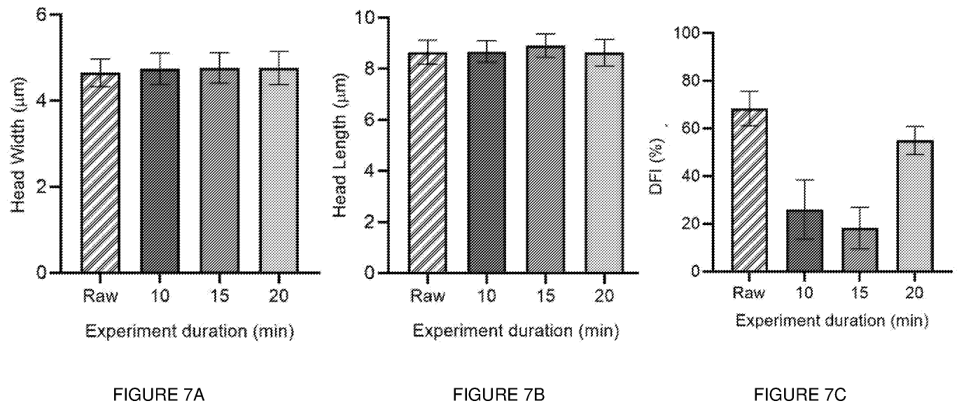

Sperm morphology and DNA integrity directly influence the fertilization rate, embryo development rate and pregnancy rate in assisted reproduction. There is also a biological relationship between sperm morphology and chromatin integrity. FIGS. 7A to 7C compare morphology and DNA integrity of selected sperm 130 with the initial semen sample 522 for experiments using bull sperm. Due to the relatively high quality of bull sperm samples to mainly include morphologically normal sperm, sperm selected from the experimental apparatus 100 also demonstrated an average head width and length of 4.8 μm and 8.7 μm, respectively (FIGS. 7A and 7B), within the range for normal morphology (a head length of 7-9 μm and a head width of 4-6 μm).

Sperm Chromatin Dispersion (SCD) assay was used to evaluate the DNA integrity of selected sperm 130 as compared with the initial semen sample 522 (FIG. 7C). SCD is a simple, reliable, and affordable method for sperm DNA integrity testing. In SCD, sperm with intact DNA forms a large halo of dispersed chromatin in agarose gel, while sperm with fragmented DNA appears without a halo or with a small halo. The ratio of sperm with fragmented DNA (large halo) over the total number of analysed sperm was expressed as the percentage of DNA fragmentation index (% DFI). The % DFI of the selected sperm after 10 minutes and 15 minutes was 26%±10% and 18%±7%, respectively, significantly improving on the DNA integrity of the initial raw sample by 62% and 74% (corresponding % DFI of 68%±7% for the initial sample). After 20 minutes of incubation time, the % DFI of selected sperm was 55%±5%, higher than selected sperm using shorter incubation times and only slightly lower than the initial semen sample 522. The achieved improvement of 74% in selected sperm DNA integrity (experiment duration of 15 minutes) considerably outperforms the current best clinical practices for sperm selection including swim-up and density gradient centrifugation. The results from testing with bull sperm indicate that an experiment duration of 15 minutes is optimal to achieve the best selection performance with respect to throughput, retrieval efficiency and DNA integrity; thus, it was used for testing the experimental apparatus 100 with human samples.

### Application to Human Sperm

To indicate the performance of the experimental apparatus 100 for translation and clinical adoption, the experimental apparatus 100 was also tested with fresh human semen samples. To evaluate the performance of the experimental apparatus 100 for oligozoospermia (low sperm count) that is a common male infertility issue, the experimental apparatus 100 was tested with diluted human samples at a concentration lower than 15 million sperm per mL.

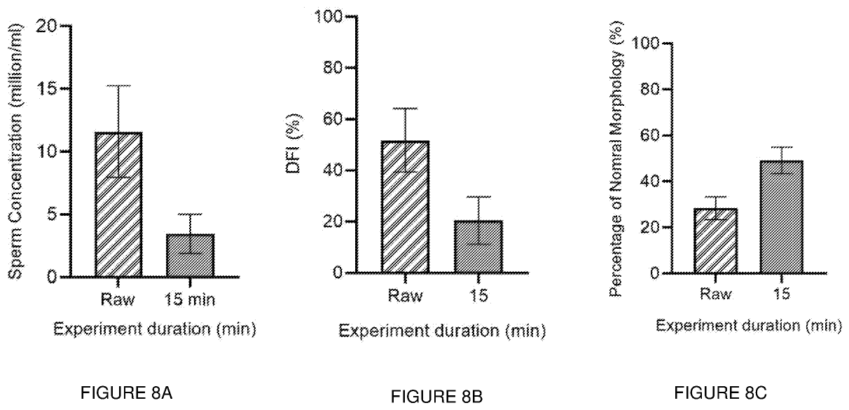

FIGS. 8A to 8C show the selected sperm concentration, DNA integrity, and morphology as compared with the initial human semen sample 522. Using diluted semen samples with an average concentration of 11.6 million per mL, 500 μL of selected sperm at a concentration of 3.5 million per mL was collected from the outlet sub-chamber 114 (FIG. 8A). It is noteworthy that the number (1,750,000) and volume of selected sperm 130 from the experimental apparatus 100 is more than sufficient to perform approximately 5 cycles of droplet-based IVF or one cycle of IUI, avoiding a major limitation of other microfluidic sperm selection technologies that only collect a small volume or number of sperm.

The % DFI of the selected human sperm was also lowered by 60% as compared with the initial human semen sample (with corresponding % DFI of 20%±9% and 52%±12%, respectively).

This improvement in DNA integrity is significant not only because the experimental apparatus 100 outperforms the current best clinical practices that achieve 50% improvement but also because it indicates the performance of the experimental apparatus 100 to improve the DNA quality of an infertile sample to a level below the fertility threshold of 30%. Human sperm morphology was also classified into a normal and three abnormal morphology classes based on WHO criteria. The percentage of sperm with normal morphology is shown in FIG. 8C. The percentage of morphologically normal sperm in the selected sperm from the experimental apparatus 100 was 49%±5%, indicating 73% improvement in morphology as compared with the initial semen sample (FIG. 8C). Taken together, experimental apparatus 100 provides a simple, reliable and high throughput approach for one-step semen purification and sperm selection in assisted reproduction. The method is capable of retrieving a sufficient number of high-quality sperm from an infertile oligozoospermia sample for direct application in IVF or IUI. The fabrication method of the experimental apparatus 100 is also simple and scalable, providing a commercially viable technology for translation and clinical adoption.

### CONCLUSION

The experiments have demonstrated the experimental apparatus 100 providing a 3D network of 560 microchannels is capable of selecting high-quality sperm with considerably improved DNA integrity and morphology through a one-step process. This 3D structure of the channel member 108 benefits from an increased contact area between the semen sample 522 and a fresh buffer fluid 520 (˜13 pL of sample per μm2) to ensure a high-throughput selection mechanism. Based on experiments with bull sperm, the experimental apparatus 100 exhibited a retrieval efficiency of 42% in under 15 minutes, considerably higher than the state-of-the-art in microfluidic sperm selection technologies. This retrieval efficiency provides a sufficient volume and number of high-quality sperm for droplet-based IVF and IUI to more frequently avoid the more invasive ICSI procedure. Testing the experimental apparatus 100 with fresh oligozoospermia human sample indicates that the experimental apparatus 100 selects sperm with 60% and 73% improvement in DNA integrity and morphology, respectively, selecting a subpopulation of healthy sperm from an infertile sample. Taken together, experimental apparatus 100 is a simple, low-cost and the rapid technology, compatible with the clinical workflow, that can improve the economics and outcome of infertility treatment worldwide.

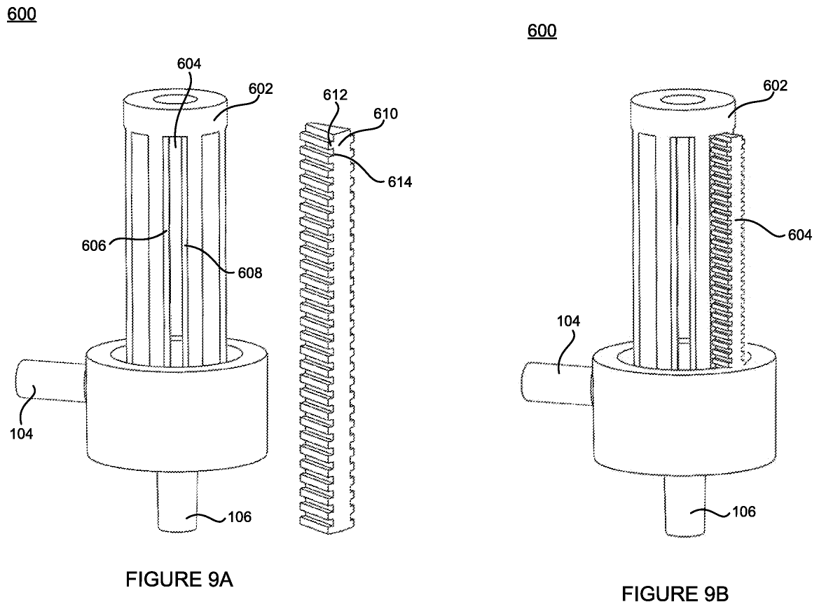

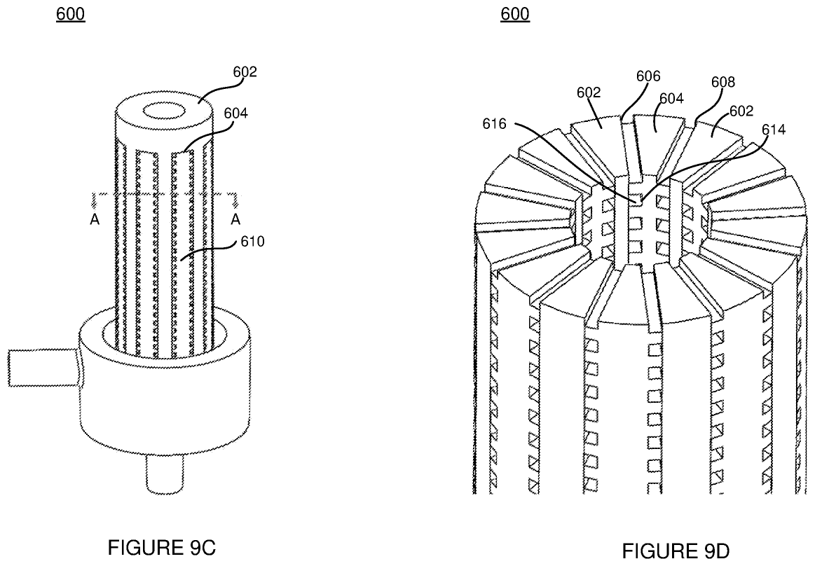

A channel member in the form of a channel assembly 600 according to an alternative embodiment is illustrated in FIGS. 9A to 9D. As more clearly shown in the partial exploded assembly view in FIG. 9A, the channel assembly 600 includes a frame 602 having a general hollow cylindrical body. The frame 602 defines a plurality of windows 604 disposed radially about a central axis of the cylindrical frame body 602. Each window 604 is elongated is a direction parallel to the central axis of the cylindrical frame body 602

The channel assembly 600 further includes a plurality of channel inserts 610 (only one shown in FIGS. 9A and 9B). Each channel insert 610 provides a plurality of protrusions 612 disposed along opposite elongate sides of the insert 610, and a plurality of grooves 614 are defined between adjacent protrusions 612. As more clearly illustrated in FIG. 9B, each channel insert 610 has an elongate body for insertion into a respective frame window 604. Corresponding opposite walls 606, 608 of the frame window 604 align and interface with the protrusions 612 of the respective channel insert 610 to define a plurality of microchannels 616 therebetween (FIG. 9D). More specifically, when the channel inserts 610 are inserted into respective windows 604 of the frame 602 as illustrated in FIGS. 9B to 9D, the protrusions 612 abut opposite walls 606, 608 of each window 604 such that a plurality of microchannels 616 are formed by the plurality of grooves 614 and walls 606, 608 of the respective windows 604 of the frame 602.

FIG. 9C illustrates the channel assembly 600 in which all channel inserts 610 are inserted into the respective windows 604 of the frame 602. As more clearly shown in the A-A cross-sectional view of the channel assembly 600 in FIG. 9D, the channel assembly 600 provides a similar structure to the channel member 108 as previously described with reference to FIG. 1C and therefore can function in accordance with the same operating principals as the channel member 108 when used in the overall apparatus 100. As such, like features of the channel assembly 600 as shown in FIG. 9D correspond to like features of the channel member 108 previously described herein.

Advantageously, the frame 602 and channel inserts 610 of the channel assembly 600 can be moulded separately for subsequent assembly to facilitate the manufacturing process, for example via injection moulding, thereby providing a useful alternative suitable for mass production.

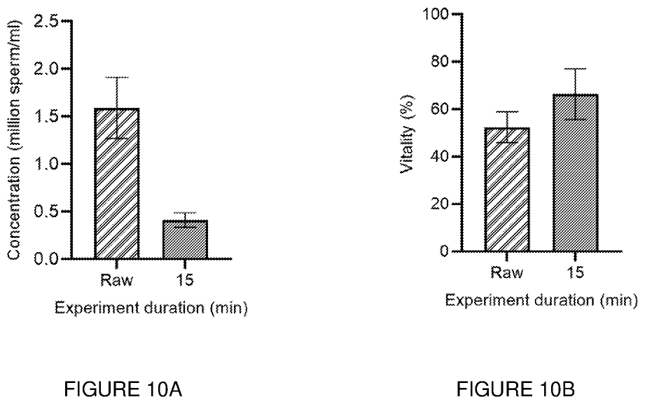

FIGS. 10A and 10B show the selected sperm concentration and percentage vitality as compared with a raw bull semen sample when an apparatus 100 having a channel assembly 600 as shown in FIGS. 9A to 9D is used. FIGS. 10A to 10B demonstrate that satisfactory results are achievable using the channel assembly 600. Using diluted semen samples with an average concentration of 1.5 million per mL, selected sperm having concentration of 0.4 million per mL was collected from the outlet sub-chamber 114 after 15 mins (FIG. 10A). As shown in FIG. 10B, the % vitality of the initial semen sample was roughly 50%, whilst the select sperm after 15 mins had a % vitality of over 65% (FIG. 10B).

### Interpretation

This specification, including the claims, is intended to be interpreted as follows:

Embodiments or examples described in the specification are intended to be illustrative of the invention, without limiting the scope thereof. The invention is capable of being practised with various modifications and additions as will readily occur to those skilled in the art. Accordingly, it is to be understood that the scope of the invention is not to be limited to the exact construction and operation described or illustrated, but only by the following claims.

The mere disclosure of a method step or product element in the specification should not be construed as being essential to the invention claimed herein, except where it is either expressly stated to be so or expressly recited in a claim.

The terms in the claims have the broadest scope of meaning they would have been given by a person of ordinary skill in the art as of the relevant date.

The terms “a” and “an” mean “one or more”, unless expressly specified otherwise.

Neither the title nor the abstract of the present application is to be taken as limiting in any way as the scope of the claimed invention.

Where the preamble of a claim recites a purpose, benefit or possible use of the claimed invention, it does not limit the claimed invention to having only that purpose, benefit or possible use.

It should be noted that terms of degree such as “generally”, “substantially”, “about” and “approximately” as used herein mean a reasonable amount of deviation of the modified term such that the end result is not significantly changed. These terms of degree should be construed as including a deviation of the modified term if this deviation would not negate the meaning of the term it modifies.

In the specification, including the claims, the term “comprise”, and variants of that term such as “comprises” or “comprising”, are used to mean “including but not limited to”, unless expressly specified otherwise, or unless in the context or usage an exclusive interpretation of the term is required.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made if the end result is not significantly changed.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The disclosure of any document referred to herein is incorporated by reference into this patent application as part of the present disclosure, but only for purposes of written description and enablement and should in no way be used to limit, define, or otherwise construe any term of the present application where the present application, without such incorporation by reference, would not have failed to provide an ascertainable meaning. Any incorporation by reference does not, in and of itself, constitute any endorsement or ratification of any statement, opinion or argument contained in any incorporated document.

1. An apparatus for separating micro-swimmers, the apparatus comprising
a chamber having an inlet sub-chamber and an outlet sub-chamber,
a plurality of microchannels disposed between the inlet sub-chamber and the outlet sub-chamber,
a passage control mechanism for controlling the passage of micro-swimmers from the inlet sub-chamber to the outlet sub-chamber via the microchannels, the passage control mechanism being configurable to operate in
a first condition in which passage of micro-swimmers from the inlet sub-chamber to the outlet sub-chamber are prevented, and
a second condition in which passage of micro-swimmers from the inlet sub-chamber to the outlet sub-chamber via the microchannels are permitted.

2. The apparatus of claim 1, wherein the micro-swimmers include sperm.

3. The apparatus of claim 1, wherein the plurality of microchannels includes a three-dimensional network of microchannels.

4. The apparatus of claim 1, the apparatus including a channel member defining the plurality of microchannels therein.

5. The apparatus of claim 4, wherein the channel member includes
a plurality of channel inserts, and
a frame defining a plurality of windows for receiving respective channel inserts therein,
each channel insert defining a plurality of external grooves to form a plurality of microchannels when the channel inserts are received in the respective windows of the frame.

6. The apparatus of claim 1, wherein the microchannels are arranged in multiple layers, each layer including a radial array of microchannels.

7. The apparatus of claim 4, wherein the channel member has a central axis and the microchannels are generally oriented perpendicularly with respect to the central axis of the channel member.

8. The apparatus of claim 4, wherein the microchannels are oriented diagonally in the channel member.

9. The apparatus of claim 1, wherein each of the microchannels are generally straight.

10. (canceled)

11. The apparatus of claim 1, wherein the passage control mechanism includes a barrier, the barrier being movable between
a first position in which the barrier prevents passage of micro-swimmers from the inlet sub-chamber to the outlet sub-chamber, and
a second position in which passage of micro-swimmers from the inlet sub-chamber to the outlet sub-chamber via the microchannels are permitted.

12. The apparatus of claim 11, wherein the barrier is located adjacent an inlet end of the microchannels in the first position, and the barrier is removed from the inlet end of the microchannels in the second position.

13. The apparatus of claim 1,
wherein passage control mechanism includes:
a first channel member defining a plurality of microchannels, and
a second channel member defining a plurality of microchannels,
wherein the first and second channel members are movable between a first position and a second position relative to one another such that:
in the first position, the microchannels in the first channel member are misaligned with the microchannels in the second channel member so as to prevent passage of micro-swimmers from the inlet sub-chamber to the outlet sub-chamber, and
in the second position, the microchannels in the first channel member are at least partially aligned with the microchannels in the second channel member so as to permit passage of micro-swimmers from the inlet sub-chamber to the outlet sub-chamber via the microchannels.

14. The apparatus of claim 13, further including
an adjustment portion associated with the first channel member, and
a pair of stoppers associated with the second channel member,
wherein movement of the adjustment portion between the pair of stoppers moves the first and second channel members between the first and second positions.

15. (canceled)

16. The apparatus according to of claim 13, including channel members defining a plurality of microchannels.

17. (canceled)

18. (canceled)

19. (canceled)

20. The apparatus of claim 1, further including:
an inlet in fluid communication with the inlet sub-chamber, and
an outlet in fluid communication with the outlet sub-chamber.

21. The apparatus of claim 1, wherein the inlet sub-chamber occupies an outer part of the chamber, and the outlet sub-chamber occupies an inner part of the chamber.

22. The apparatus of claim 21, wherein the outlet sub-chamber occupies a centre portion of the chamber, and the inlet sub-chamber occupies an outer part of the chamber adjacent a periphery of the chamber.

23. The apparatus of claim 1, wherein each microchannel has an inlet end adjacent the inlet sub-chamber and an outlet end adjacent the outlet sub-chamber such that micro-swimmers moving from the inlet sub-chamber to the outlet sub-chamber passes through the microchannels from the respective inlet ends to the outlet ends, and wherein a distance between the inlet ends of the microchannels and an inner peripheral surface of the chamber defines a thickness of the inlet sub-chamber, and wherein the thickness of the inlet sub-chamber is generally between 0.2 mm to 6 mm.

24. The apparatus of claim 20, further including a transfer member movable within the outlet sub-chamber for transferring micro-swimmers from the outlet sub-chamber to the outlet.

25. (canceled)

26. The apparatus of claim 1, wherein the total number of microchannels is between 60 and 70,000.

27. (canceled)

28. (canceled)

29. (canceled)

30. A method of separating micro-swimmers using the apparatus of claim 1, the method including the steps of
loading a buffer fluid into the chamber,
configuring the passage control mechanism in the first condition,
loading a sample of micro-swimmers into the inlet sub-chamber,
configuring the passage control mechanism in the second condition, and
collecting the micro-swimmers from the outlet sub-chamber.

31-35. (canceled)

36. A method of separating micro-swimmers, the method including the steps of
loading a buffer fluid into a reservoir, the reservoir having an inlet reservoir and an outlet reservoir,
sealing the inlet reservoir,
loading a sample of micro-swimmers into the inlet reservoir,
directing passage of micro-swimmers from the inlet reservoir to the outlet reservoir via a plurality of microchannels, and
collecting the micro-swimmers from the outlet reservoir.

37-42. (canceled)
